(19) United States
(12) Patent Application Publication
(10) Pub. No.: US 2009/0312716 A1
(43) Pub. Date: Dec. 17, 2009

(54) MEDICATION CARTRIDGE PISTON

(76) Inventors: Ari Tao Adler, Cambridge, MA (US); James J. Collins, JR., Carmel, IN (US); Jose Colucci, JR., Lexington, CA (US); Jared Alden Judson, Topsfield, MA (US)

Correspondence Address: ELI LILLY & COMPANY, PATENT DIVISION, P.O. BOX 6288, INDIANAPOLIS, IN, 46206-6288, US

(21) Appl. No.: 12/307,788

(22) PCT Filed: Jun. 27, 2007
(86) PCT No.: PCT/US07/72173

§ 371 Date: Jan. 7, 2009

Related U.S. Application Data
(60) Provisional application No. 60/807,003, filed on Jul. 11, 2006.

(51) Int. Cl. A61M 5/315

(52) U.S. Cl. 604/222

(57) ABSTRACT

A medication cartridge piston of a cartridge for an injection device. In one form, the piston includes a body (32) formed of a first material and including an interior hollow with a rearward facing mouth allowing for insertion of a drive member of the injection device. The body includes a first portion and a second portion disposed rearwared of the first portion, a sealing gland (34) secured to the body and formed of a material that is less rigid than the first material, the sealing gland ringing at least an axial segment of the second portion and having at least one exterior periphery region for sealingly engaging an interior surface of the cartridge barrel, and the body first portion including a thrust surface (4S) within the interior hollow for direct abutment by the drive member, at least a portion of the thrust surface disposed forward of a forward edge of a forwardmost sealing gland exterior periphery region of the at least one exterior periphery region. This piston can be operatively pulled by the drive member in a forward direction within the cartridge barrel to dispense medication from the cartridge. In an alternate embodiment, a piston is provided which is adapted for use with different drive members of injection devices.

### BACKGROUND OF THE INVENTION

The present invention pertains to medication cartridges, and, in particular, to pistons of medication cartridges.

A wide variety of medication injection devices are available which allow people, such as patients or health care professionals, to administer pharmaceuticals to themselves or others. Many of these devices include or use cartridges, which cartridges each hold one or more doses of the desired pharmaceutical that can be forced from the cartridge by selective advancement of a sealing piston of the cartridge.

Existing cartridge pistons are effective but are not without their shortcomings. For one thing, standard pistons made entirely of a resilient material may have limitations related to dose accuracy, or delivery time, or glide force requirements. Another known piston that includes a rigid core and is disclosed in International Publication Number 2005/099793 may have glide force limitations.

Thus, it would be desirable to provide an improved cartridge piston that provides one or more advantages over medication cartridge pistons of the prior art.

### BRIEF SUMMARY OF THE INVENTION

In one form thereof, the present invention provides a medication cartridge piston of a cartridge for an injection device. The piston is advanceable in a forward direction within a barrel of the cartridge for dispensing medication from the cartridge. The piston includes a body formed of a first material and including an interior hollow with a rearward facing mouth allowing for insertion of a drive member of the injection device, which body includes a first portion and a second portion disposed rearward of the first portion, a sealing gland secured to the body and formed of a material that is less rigid than the first material, the sealing gland ringing at least an axial segment of the second portion and having at least one exterior periphery region for sealingly engaging an interior surface of the cartridge barrel, and the body first portion including a thrust surface within the interior hollow for direct abutment by the drive member, at least a portion of the thrust surface disposed forward of a forward edge of a forwardmost sealing gland exterior periphery region of the at least one exterior periphery region.

In another form thereof, the present invention provides a medication cartridge piston of a cartridge for an injection device, which piston is advanceable in a forward direction within a barrel of the cartridge for dispensing medication from the cartridge. The piston includes a body formed of a first material and including an interior hollow with a rearward facing mouth allowing for insertion of a drive member of the injection device, the body including a first portion and a second portion disposed rearward of the first portion, a sealing gland secured to the body and formed of a material that is less rigid than the first material, the sealing gland ringing at least an axial segment of the first portion and having at least one exterior periphery region for sealingly engaging an interior surface of the cartridge barrel, the body including a thrust surface within the interior hollow for direct abutment by the drive member, the body second portion disposed rearward of a rearwardmost sealing gland exterior periphery region of the at least one exterior periphery region, the second portion having at least one section for slidably contacting without sealing the cartridge barrel interior surface for piston centering, and the body second portion further including at least one ledge rearward of the thrust surface and which extends radially inward to define an abutment shoulder within the interior hollow, which shoulder is sized to define a central opening to allow free passage of the drive member of the injection device which abuts the thrust surface, and which shoulder is sized for axial abutment by an enlarged foot of a drive member of a different injection device.

One advantage of the present invention is that a medication cartridge piston may be provided which in effect may be pulled through a cartridge by a drive member, thereby possibly offering more dynamic stability and reduced friction.

Another advantage of the present invention is that a medication cartridge piston may be provided which uses a limited amount of resilient sealing material, which may improve piston performance related to one or more of dose accuracy, time for full dose delivery, and glide force, and which also may allow accurate axial positioning in a cartridge for filling.

Yet another advantage of the present invention is that a medication cartridge piston may be provided which functions with differently designed drive members.

### BRIEF DESCRIPTION OF THE DRAWINGS

The above-mentioned and other advantages and objects of this invention, and the manner of attaining them, will become more apparent, and the invention itself will be better understood, by reference to the following description of embodiments of the invention taken in conjunction with the accompanying drawings, wherein:

FIG. 1 is a front perspective view of a cartridge with a piston of the present invention;

FIG. 2 is a front perspective view of the cartridge and piston of FIG. 1, with the piston shown removed from the cartridge barrel and in an exploded state;

FIG. 3 is a side view of the piston of FIG. 2 in an assembled state;

FIG. 4 is a bottom, longitudinal cross-sectional view of the piston of FIG. 3 shown installed in the partially shown cartridge barrel;

FIG. 5 is a view of the piston of FIG. 4 while being engaged by an advancing drive member of an injection device with which the invention may find advantageous application; and

FIG. 6 is a longitudinal cross-sectional view of another piston of the present invention shown installed in a partially shown cartridge barrel and being engaged by a different advancing drive member of an injection device.

Corresponding reference characters indicate corresponding parts throughout the several views. Although the drawings represent embodiments of the present invention, the drawings are not necessarily to scale, and certain features may be exaggerated or omitted in some of the drawings in order to better illustrate and explain the present invention.

### DETAILED DESCRIPTION OF THE INVENTION

Referring now to FIG. 1, there is shown a cartridge with one embodiment of a sealing piston of the present invention. Other than the piston disposed therein, the cartridge is generally of a known construction, and further does not form a part of the present invention. The cartridge, generally designated 20, includes a tubular body made of glass having a barrel portion 22, and a reduced-diameter neck portion 24 at the forward end of barrel 22. The cartridge also includes a needle-piercable septum 26 that is secured in a fluid-tight manner over the forward, outlet end of the cartridge body by an aluminum cap 28 that is crimped over a radially enlarged forward end of the neck portion 24. Cartridge 20 may be used in injection devices of various designs, such as injection pens, which devices have a drive member insertable within the cartridge barrel so as to advance the piston as further described below.

For the shown cartridge with piston, front and rear, and forward and rearward, refer to relative locations, wherein a direction of travel of the piston within the cartridge body during use to empty the medication from the cartridge is considered a forward travel. These references, as well as any other directional references in this detailed description with respect to the Figures, such as side or bottom, are intended for convenience of description, and by itself do not limit the present invention or any of its components to any particular positional or spatial orientation.

A sealing piston according to one form of the present invention is shown in FIG. 1 disposed within the barrel of cartridge 20 and is generally designated 30. Piston 30 is axially slidably and sealably engaged with the barrel interior wall 23 and serves to define the rearward end of the medication filled, variable volume reservoir 25 of the cartridge 20. Piston 30 is generally circular in transverse cross-section to conform to the cylindrical tubular barrel of the shown cartridge, but may be otherwise shaped, such as to account for different cross-sectional cartridge barrel shapes.

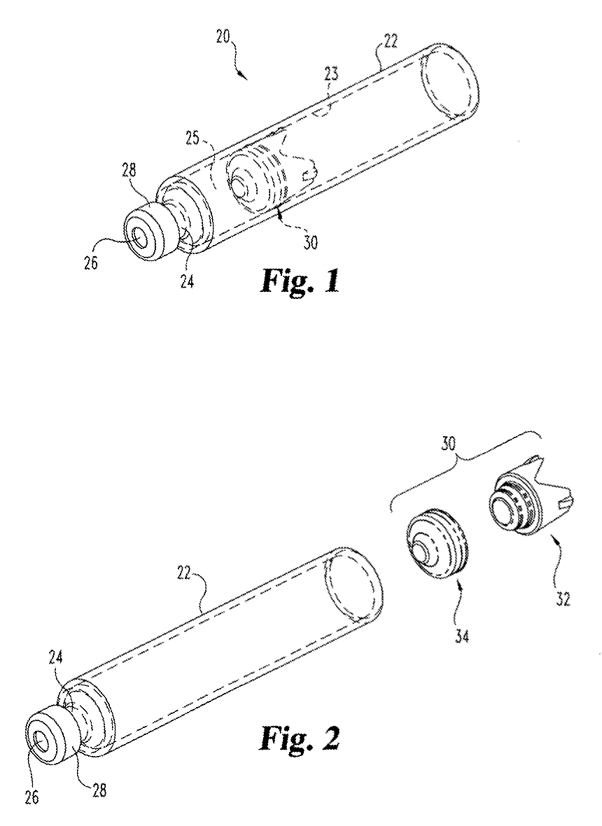

As further shown in FIG. 2, piston 30 consists of two separately formed pieces in the shown embodiment, namely a piston core 32 and a piston gland 34. The core and gland are fixedly connected together during piston manufacture to remain as a unit throughout the useful life of the piston.

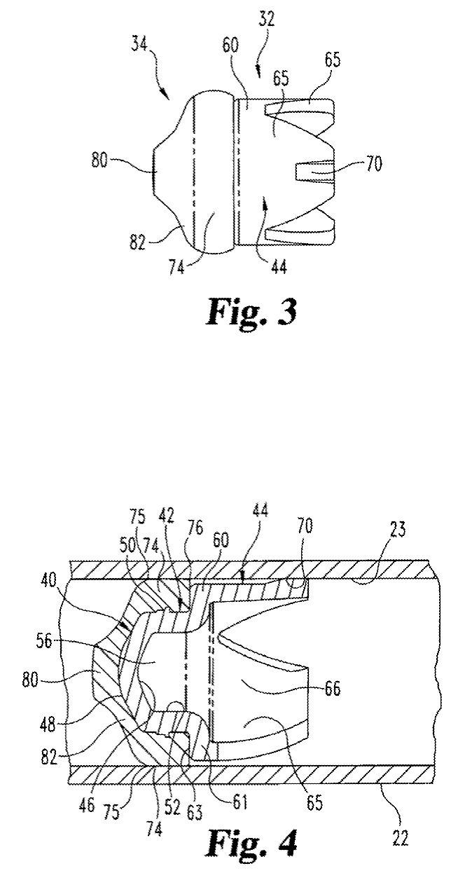

With additional reference to FIGS. 3 and 4, piston core 32 is a rigid body formed of a non-compliant plastic or other suitably stiff material. The core body generally includes a forward portion 40, a central portion 42, and a rearward portion 44.

Forward portion 40 is solid, that is without holes, and is symmetrical about the longitudinal axis of the piston. Forward portion 40 is a bowed disc in shape, which bowing results in a concave rear surface 46 and a convex forward surface 48. Other concave and convex shapings, such as, for example, a more conical surface or a faceted surface, alternatively may be used. The radially outer region of forward portion 40 transitions to the forward edge of body central portion 42 that is tubular, solid and generally cylindrical in shape.

A radially outwardly projecting barbed lip 50 rings a middle region of tubular portion 42 to aid in retaining gland 34. The cylindrical interior surface 52 of body central portion 42 and the concave rear surface 46 of body forward portion 40 define an interior hollow or blind bore 56 having a rearward facing mouth into which a drive member of an injection device can freely be inserted and withdrawn.

Rearward portion 44 in its entirety projects rearward of sealing gland 34 and serves to center and stabilize piston 30 within the cartridge barrel when in engagement with barrel surface 23. Rearward portion 44 includes a solid, annular segment 60 with a shoulder region 61 that extends radially outward from the rearward end of central portion 42. The open interior 66 of the rearward portion is in axial alignment with hollow 56. A forward facing surface 63 of shoulder region 61 is a support surface for sealing gland 34. Segment 60 is in radially spaced relationship with barrel surface 23.

A plurality of flanges or fingers 65 of rearward portion 44 extend rearward from segment 60. The shown plurality of flanges 65 include three, equally angularly spaced flanges disposed at the radial periphery of segment 60. Each flange 65, proximate its rearward end, includes a glide boss 70 that radially protrudes from the outer periphery of the flange. Each glide boss 70 in intended to slidingly engage the interior surface 23 of barrel 22 at all times to maintain the centering of the piston within the barrel. The flange shape and construction provides a resiliency to flanges 65. This resilience accounts for the ability of the piston to be inserted into the cartridge barrel even if the opening into that barrel is smaller than its internal diameter, such as due to a bead or rim formed at the cartridge barrel rearward end, and well as accounts for tolerances in the barrel interior diameter. The small surface area of each glide boss 70 in contact with the cartridge barrel limits the frictional resistance from the core that impacts glide force.

While the core rearward portion 44 is shown having three flanges 65, fewer including zero such flanges, or additional such flanges, may be provided within the scope of the invention. Still further, such as in alternate embodiments in which only some or none of the flange regions are to be in continuous contact with the barrel surface 23, the glide bosses may be eliminated or reduced in number to as few as one.

Piston gland 34 is formed in one piece from a material compatible with the medication to be contained in the cartridge in which it is used, but which material is more resilient and less stiff than the material of core 32 so as to form a suitable fluid-tight seal with the barrel. One such material may be butyl rubber. Piston gland 34 is fixedly secured to core 32 during assembly using a suitable technique, such as by fit, bond or multi-shot injection molding. Piston gland 34 is shaped in conjunction with the piston core geometry to be self energizing. Piston gland 34 includes a sealing ring region 74 that circumferentially extends around core central portion 42. As shown in FIG. 4, along its entire axial length from its forward edge 75 to its rearward edge 76, the outer periphery of sealing ring region 74 is in direct sealing and slidable engagement with interior surface 23 of cartridge barrel 22 when piston 30 is oriented centrally therein so as to not be in a canted orientation relative to the axial direction. Although only one sealing ring region 74 is shown, multiple axially spaced sealing ring regions, possibly formed as separate O-rings that are then assembled to the core, may be employed within the scope of the invention.

Piston 30 is designed such that during advancement by a drive member of an injection device with which cartridge 20 is particularly adapted for use, the piston core is directly and drivingly abutted by that drive member. This driving contact, which happens without requiring any attachment to the drive member such that the drive member can be retracted without any detachment from or pulling rearward movement of the piston, occurs at least in part at a point forward of the forward edge 75 of sealing ring region 74, and may occur entirely forward of that forward edge 75, but may be of such a size or shape to also extend rearward of that forward edge. In the shown embodiment, this contact forward of the sealing edge 75 takes place at the forwardmost area of concave rear surface 46, which contacted area serves as a thrust surface against which the complementarily rounded end of the device drive member of body abuts to provide a driving force. As such, the piston gland 32 is operatively pulled, rather than pushed, forward by the drive member during advancement of piston 30 to force medication from cartridge 20.

Plunger gland 34 is shown completely covering core forward portion 40 by way of a nipple region 80 and a spanning region 82. In alternate embodiments in which the core forward portion is formed of either a suitable material or covered with, for example, another medication compatible material, the sealing gland need not cover the forward portion. Nipple region 80 is made by a thickened layer of material which forms a protruberance centered on the end of core forward portion 40. Nipple region 80 is integrally connected with the sealing ring region 74 by the annular spanning region 82. Nipple region 80 and spanning region 82 are shaped to match the interior surface of the neck 24 of the cartridge in order to more fully inject the cartridge contents so as to reduce wastage of medication. All of spanning region 82 is in radially spaced arrangement with the interior surface 23 of the barrel 22 to be free of contact therewith when the piston is in its centered orientation, in contrast to sealing ring region 74. Spanning region 82 contacts the neck 24 when the cartridge contents are spent. Although spanning region 82 is shown having an outer radial region that is rounded to provide a convex lead-in to forward edge 75 of sealing ring region 74, such convex lead-in may be replaced with different shapes, including a straighter lead-in or a concave lead-in, which replacement shapes may be more suitable when the medication within the cartridge is a suspension rather than a solution.

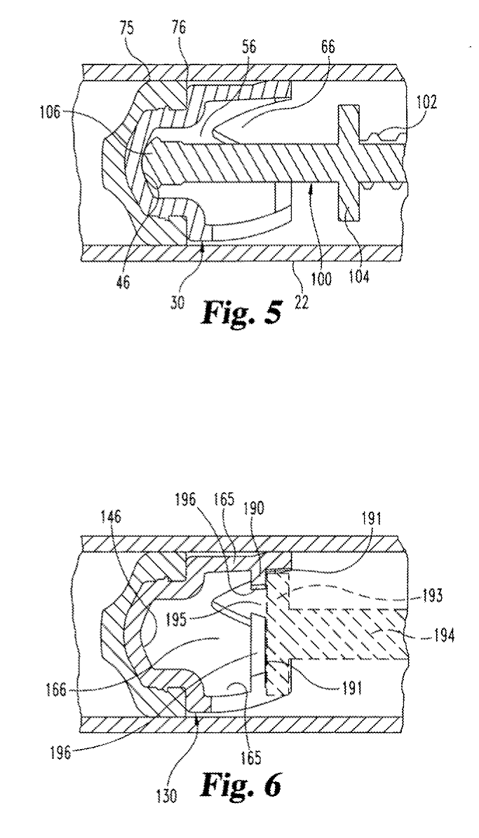

One drive member with which piston 30 is particularly suited for use is shown in part in FIG. 5. The drive member is designated 100 and is shown including threads 102 that can be engaged with a not shown drive mechanism of the injection device, such as a reusable injection pen, which screws the drive member forward during advancement. The inventive piston 30 may also be advanced by a non-rotating drive member. A transversally extending collar 104 of drive member 100 may serve to limit the manual pushback of the lead screw when a new cartridge is loaded in the device. The forward end 106 of drive member 100 is shown having a convex surface shape with a smaller radius of curvature than the concavity of rear surface 46. This design, which provides a suitable bearing surface or interface therebetween, allows relative rotation of the shown drive screw to the piston and cartridge, which piston is not rotatably engaged by the rotating drive member. For the rounded rear surface and drive member tip shown, different radii may be employed, such as drive member radii which are equal to or other smaller values than that of the rear surface. Larger radii of the drive member tip than the rear surface, or non-concave or non-convex shapes for either or both the rear surface and drive member tip, may be provided in alternate embodiments, with a more pulling type advancement of the piston through the cartridge being achieved with such embodiments if the bearing surface is at least in part forward of sealing edge 75.

Referring now to FIG. 6, there is shown an alternate embodiment of a piston of the present invention. In this embodiment, the piston, generally designated 130, is identical in all respects to piston 30 except as it relates to the provision of a ledge surface intended to allow engagement by a drive member different from that shown in FIG. 5. In particular, at least one flange 165, and preferably all of the flanges 165, includes a ledge or lip 190 that radially protrudes inward into the interior hollow 166 of the piston core. The rearward facing surface 191 of each ledge 190 is disposed at a common axial location of the piston longitudinal axis, and further is shown oriented transverse to that axis. This axial location can be selected such that after assembly and filling of a cartridge that utilizes piston 130, surface 191 is disposed in that cartridge at the same axial location as the rear face of a conventional piston in a similar cartridge, thereby providing the flexibility of using a cartridge with piston 130 instead of a more conventional cartridge/piston.

The ledge surfaces 191 define an abutment shoulder or thrust surface that can be directly engaged by the enlarged foot of a drive member of some existing injection devices, which foot and drive member move axially as a unit and are abstractly shown in dashed lines in cross-section at 193 and 194, respectively. Although abstractly shown integrally formed, foot 193 and drive member 194 may be separate pieces coupled together, thereby allowing the drive member 194 to be screwed out by the injection device further into the cartridge barrel while the foot 193 remains rotatably stationary with piston 130.

The ledge surfaces 191 are sized such the central opening 195 defined by the radially inward faces 196 of ledges 190 is sufficiently large to allow free passage of the narrower type of drive member shown in FIG. 4 which would engage thrust surface 146. As a result, the medication cartridge equipped with piston 130 can be used with injection devices having, for example, either an existing drive member with foot as shown in FIG. 6, or a slim drive member such as shown in FIG. 4.

Although piston 130 includes three flanges 165 each with a ledge 190, in alternate embodiments of this type of piston, and provided a suitable thrust surface is still provided, not every flange needs to include a ledge. And further, with respect to this type of piston that includes no flanges but instead includes a uninterrupted rearward portion, one or more ledges 190 can be provided around the interior periphery of the rearward portion to achieve the desired thrust surface.

In addition, although surfaces 191 are shown exactly transverse or perpendicular to the axial direction in FIG. 6, in order to promote passage of a drive member shown in FIG. 4, such surfaces may be sloped slightly forward and/or have a chamfered inner edge, whereby a drive member of the type shown in FIG. 4 that at first bumps such surface during its insertion will be guided into and through the central opening 195.

In still another embodiment that is not shown, the inventive piston is basically the same as piston 130 but differs in that its thrust surface directly engageable by the drive member of FIG. 4 is positioned more rearward. In particular, none of the thrust surface is located forward of the forward edge 75 of the forwardmost sealing region 74. For such embodiment, the thrust surface may, but is not required to, be located at least in part, and possibly entirely, forward of the rearward edge of the rearwardmost sealing region, such as the rearward edge 76 of sealing ring region 74 in the sealing gland design of the first embodiment. This embodiment advantageously employs the feature of the embodiment shown in FIG. 5 which accommodates the drive members shown in FIGS. 4 and 6, but does not employ the piston pulling feature of the first embodiment.

While this invention has been shown and described as having preferred designs, the present invention may be modified within the spirit and scope of this disclosure. This application is therefore intended to cover any variations, uses or adaptations of the invention using its general principles. Further, this application is intended to cover such departures from the present disclosure as come within known or customary practice in the art to which this invention pertains.

We claim:

1. A medication cartridge piston of a cartridge for an injection device, the piston advanceable in a forward direction within a barrel of the cartridge for dispensing medication from the cartridge, the piston comprising:
a body formed of a first material and including an interior hollow with a rearward facing mouth allowing for insertion of a drive member of the injection device, said body including a first portion and a second portion disposed rearward of said first portion;
a sealing gland secured to said body and formed of a material that is less rigid than said first material, said sealing gland ringing at least an axial segment of said second portion and having at least one exterior periphery region for sealingly engaging an interior surface of the cartridge barrel; and
said body first portion including a thrust surface within said interior hollow for direct abutment by the drive member, at least a portion of said thrust surface disposed forward of a forward edge of a forwardmost sealing gland exterior periphery region of said at least one exterior periphery region.

2. The medication cartridge piston of claim 1 wherein said body comprises a third portion disposed rearward of a rearwardmost sealing gland exterior periphery region of said at least one exterior periphery region, said third portion having at least one section for slidably contacting without sealing the cartridge barrel interior surface for piston centering.

3. The medication cartridge piston of claim 2 wherein said body third portion further comprises at least one ledge that extends radially inward to define an abutment shoulder within said interior hollow, said shoulder sized to define a central opening to allow free passage of the drive member of the injection device which abuts said thrust surface, said shoulder sized for axial abutment by an enlarged foot of a drive member of a different injection device.

4. The medication cartridge piston of claim 2 wherein said body third portion comprises a plurality of axially extending flanges with rearward tips that are angularly spaced from each other.

5. The medication cartridge piston of claim 4 wherein said body third portion further comprises a plurality of ledges that extend radially inward from said flanges so as to define a shoulder within said interior, said shoulder sized to define a central opening to allow free passage of the drive member of the injection device which abuts said thrust surface, said shoulder sized for axial abutment by an enlarged foot of a drive member of a different injection device.

6. The medication cartridge piston of claim 4 wherein said at least one section comprises at least one radially outwardly protruding boss on at least one of said plurality of axially extending flanges.

7. The medication cartridge piston of claim 6 wherein said at least one radially outwardly protruding boss comprises a boss on each of said plurality of axially extending flanges.

8. The medication cartridge piston of claim 1 wherein said thrust surface is concave.

9. The medication cartridge piston of claim 1 wherein said sealing gland further covers all of a forward face of said body first portion.

10. The medication cartridge piston of claim 1 wherein said sealing gland comprises a one-piece construction.

11. A medication cartridge piston of a cartridge for an injection device, the piston advanceable in a forward direction within a barrel of the cartridge for dispensing medication from the cartridge, the piston comprising:
a body formed of a first material and including an interior hollow with a rearward facing mouth allowing for insertion of a drive member of the injection device, said body including a first portion and a second portion disposed rearward of said first portion;
a sealing gland secured to said body and formed of a material that is less rigid than said first material, said sealing gland ringing at least an axial segment of said first portion and having at least one exterior periphery region for sealingly engaging an interior surface of the cartridge barrel;
said body including a thrust surface within said interior hollow for direct abutment by the drive member;
said body second portion disposed rearward of a rearwardmost sealing gland exterior periphery region of said at least one exterior periphery region, said second portion having at least one section for slidably contacting without sealing the cartridge barrel interior surface for piston centering; and
said body second portion further comprising at least one ledge rearward of said thrust surface and which extends radially inward to define an abutment shoulder within said interior hollow, said shoulder sized to define a central opening to allow free passage of the drive member of the injection device which abuts said thrust surface, said shoulder sized for axial abutment by an enlarged foot of a drive member of a different injection device.

12. The medication cartridge piston of claim 11 wherein said body second portion comprises a plurality of axially extending flanges with rearward tips that are angularly spaced from each other, and wherein said at least one ledge comprises a plurality of ledges that extend radially inward from said flanges.

13. The medication cartridge piston of claim 12 wherein said at least one section comprises at least one radially outwardly protruding boss on at least one of said plurality of axially extending flanges.

14. The medication cartridge piston of claim 13 wherein said at least one radially outwardly protruding boss comprises a boss on each of said plurality of axially extending flanges.

15. The medication cartridge piston of claim 11 wherein at least a portion of said thrust surface is disposed forward of a rearward edge of a rearwardmost sealing gland exterior periphery region of said at least one exterior periphery region.

16. The medication cartridge piston of claim 11 wherein at least a portion of said thrust surface is disposed forward of a forward edge of a forwardmost sealing gland exterior periphery region of said at least one exterior periphery region.
